SEQUENCE LISTING (6 sequences)

SEQ ID NO: 1 - DNA, 19 bp, artificial sequence
  primer Fwd_AGN
tctaccgctgcgaggtgat

SEQ ID NO: 2 - DNA, 22 bp, artificial sequence
  primer Rev_AGN
tgtaatggaacacgatgccttt

SEQ ID NO: 3 - DNA, 20 bp, artificial sequence
  primer Fwd_Col2A
actggattgaccccaaccaa

SEQ ID NO: 4 - DNA, 22 bp, artificial sequence
  primer Rev_Col2A
tccatgttgcagaaaaccttca

SEQ ID NO: 5 - DNA, 21 bp, artificial sequence
  primer Fwd_Col1A
gctacccaacttgccttcatg

SEQ ID NO: 6 - DNA, 24 bp, artificial sequence
  primer Rev_Col1A
ttcttgcagtggtaggtgatgttc

(19) United States
(12) Patent Application Publication
(10) Pub. No.: US 2014/0349330 A1
(43) Pub. Date: Nov. 27, 2014

(54) METHODS FOR AGGREGATION AND DIFFERENTIATION OF MAGNETIZED STEM CELLS

(71) Applicants: Delphine Fayol, Montrouge (FR); Nathalie Luciani, Conflans-Saint-Honorine (FR); Catherine Le Visage, Paris (FR); Florence Gazeau, Le Kremlin Bicetre (FR); Claire Wilhelm-Hannetel, Velizy (FR)

(72) Inventors: Delphine Fayol, Montrouge (FR); Nathalie Luciani, Conflans-Saint-Honorine (FR); Catherine Le Visage, Paris (FR); Florence Gazeau, Le Kremlin Bicetre (FR); Claire Wilhelm-Hannetel, Velizy (FR)

(73) Assignee: CENTRE NATIONAL DE LA RECHERCHE SCIENTIFIQUE, PARIS CEDEX 16 (FR)

(21) Appl. No.: 14/241,672

(22) PCT Filed: Sep. 3, 2012
(86) PCT No.: PCT/EP2012/067048

§ 371 Date: Jul. 1, 2014

(30) Foreign Application Priority Data
Sep. 1, 2011 (FR) 1157755

(51) Int. Cl. C12N 13/00; C12N 5/077

(52) U.S. Cl. CPC C12N 13/00; C12N 5/0655; C12N 2506/02; C12N 2506/1346; C12N 2501/15
USPC 435/29; 435/173.9; 435/377

(57) ABSTRACT

The invention relates to a process which enables optimal aggregation of cells, typically of stem cells, promoting the organisation thereof and advantageously the differentiation thereof, in particular in the context of the formation of a tissue substitute. This process comprises exposing pretreated cells to a magnetic field and makes it possible to obtain large cell aggregates, even prepared in the absence of support matrix and/or of growth factor. The invention also relates to the cell aggregates that can be obtained using such a process and also to the uses thereof as tissue initiators with a view to obtaining a tissue structure of interest in vitro, ex vivo or in vivo. Moreover, it relates to the resulting tissue structures and to the uses thereof in research or in therapy as tissue substitutes. The present application also provides a method which advantageously makes it possible to monitor the development of the tissue of interest in vivo.

### FIELD OF THE INVENTION

The invention relates to the field of cell therapy, regenerative medicine and tissue engineering. It relates more particularly to a process which enables optimal aggregation of cells, typically of stem cells, promoting the organisation thereof and advantageously the differentiation thereof, in particular in the context of the formation of a tissue substitute. This process comprises exposing pretreated stem cells to a magnetic field and makes it possible to obtain cell aggregates comprising differentiated cells, in particular large cell aggregates. Surprisingly and contrary to current practice, this aggregation process can advantageously be carried out in the absence of exogenous three-dimensional cell adhesion support matrix and/or of growth factor. The invention also relates to the cell aggregates which can be obtained using such a process and also to the uses thereof as tissue initiators with a view to obtaining a tissue structure of interest in vitro, ex vivo or in vivo. Moreover, it relates to the resulting tissue structures and to the uses thereof in research or in therapy as tissue substitutes. The present application also provides a method which advantageously makes it possible to monitor the development of the tissue of interest in vivo.

### PRIOR ART

The cell aggregation method commonly used in tissue engineering consists in centrifuging said cells. However, this method makes it possible to obtain only small aggregates (approximately 1 mm³) containing a limited number of cells (at most approximately 250,000 cells). Even if the number of cells centrifuged is increased, it is not possible at the current time to obtain aggregates of greater volume, since the cells present at the heart of the aggregate necrose (probably because of the restricted access to nutrients and to oxygen).

The limited size of the aggregates obtained is not without its problems for routine clinical tissue substitute implantations. This constraint in fact imposes the reconstitution of a tissue initiator which has a critical size before implantation, by placing the small cell aggregate(s) in culture ex vivo, or the implantation of a very high number of cell aggregates to fill a lesion (variable area which can commonly reach 16 cm² in the case of joint cartilage lesions). Such an implantation, in addition to making the manipulation by the surgeon more difficult, is often responsible for problems regarding retention of said initiators at the site of implantation such that only a small number of the implanted cells actually contribute to the tissue regeneration. The use of a support matrix for filling the lesion at the implantation site and promoting retention of the cell aggregates very often proves to be essential in this context.

The implantation of cell aggregates is a therapeutic strategy used in particular for treating pathological cartilage conditions. Cartilage plays an essential role in impact resistance, but has a very limited capacity to regenerate spontaneously after a lesion (traumatic impacts, arthrosis, etc.). It consists of an extracellular matrix which gives it its mechanical strength, and of cells, chondrocytes, which are responsible for the synthesis of this matrix.

Pathological cartilage conditions, in particular pathological joint cartilage conditions, are at the current time treated by means of purely surgical techniques (microfracture) or by means of techniques which include chondrocyte implantation [osteochondral transplantation (OATS or mosaicplasty), autologous chondrocyte implantation/transplantation ACI/ACT].

Microfracture makes it possible to generate an influx of blood which will stimulate a healing response. However, the results obtained are mixed and the newly formed cartilage, which is fibrous in nature, subsequently degrades (often within six to twelve months). This method is nevertheless used by a number of orthopaedic surgeons since it is simple to implement. It applies to young subjects (in particular athletic subjects) but is not suitable for people suffering from arthrosis in whom the cartilage regeneration capacities are reduced.

Osteochondral transplantation consists of allografts or autografts of cartilage and of underlying bone (a bone graft attaches quite naturally to another piece of bone, whereas it is necessary to suture cartilage, with a not insignificant risk of detachment of the latter). In the latter case, cartilage is taken from areas where the load is low so as to be reimplanted in areas where the load is high. The main problems encountered are morbidity at the donor site (destruction of healthy cartilage) and incomplete and temporary regeneration owing to degeneration of the graft which is not adapted to the mechanical stresses of the implantation site.

In the case of allografts (grafts taken from corpses and optionally stored after freezing or lyophilisation), good clinical results have been obtained in the long term (10 years). The cartilage is sparingly subject to rejection phenomena. The main limitations are poor graft availability and the risk of disease transmission.

The implantation of autologous chondrocytes, which has been possible since 1968, is preceded by a biopsy of healthy cartilage (Carticel® from the company Genzyme, ACI-Maix® from the company Matricel). The chondrocytes are extracted and amplified in vitro (2D) before being implanted. A piece of tissue, sutured at the level of the defect, ensures that the cells remain located at the implantation site. However, the implanted tissue ends up degrading just as in the case of autografts.

The approaches more recently explored for the engineering of cartilaginous tissues consist (i) of the use of porous matrices intended to promote, after they have been implanted in the patient, the migration of the native differentiated cells of said patient (U.S. Pat. No. 6,179,871; Chondrotissue® from the company BioTissue Technologies; Robert et al., Biomaterials 31, 2010), or of matrices combined with cells, the action of which must be to promote and guide the tissue regeneration after the amplification in 2D cell culture of said cells (MACI® from the company Genzyme), (ii) of the implementation of a method involving a step of culture in suspension (US 2005/0074477) and (iii) of the implantation of mesenchymal stem cells or MSCs (at the clinical trial stage).

The latter approach constitutes a variation of chondrocyte implantation. However, it has the same problem of cell retention at the implantation site, added to which are those associated with the differentiation of the MSCs.

In this type of procedure, the MSCs are i) implanted without prior treatment, ii) implanted after having been activated by proteins, the role of which is to programme them to become differentiated cells, or iii) differentiated in vitro and implanted in the form of tissue initiators.

In this respect, the “cell aggregation by centrifugation” technique enables the in vitro differentiation of the stem cells, such as mesenchymal stem cells (MSCs), into chondrocytes capable of producing the extracellular matrix of the cartilaginous tissue (this differentiation process is also called chondrogenesis). The centrifugation of MSCs at 200 g for 5 minutes, followed by culturing of the cell aggregate obtained in the presence of specific soluble chondrogenesis-inducing factors for 14 days (cf. Schäler et al., Radiology: vol. 211, No. 2, 2007) or 28 days, in particular in the presence of TGFβ1, TGFβ2, TGFβ3 or BMP-2, are examples of implementation of the cell aggregation by centrifugation technique.

However, when applied to stem cells intended to differentiate into cartilaginous cells, the method of cell aggregation by centrifugation causes the formation of a fibrous shell around the cell aggregate limiting, in the context of the clinical use of said aggregate as a tissue initiator, its ability to integrate into the native cartilaginous tissue of the recipient subject and promoting its subsequent degradation.

The inventors presently propose a solution to the many problems encountered during the implementation of the techniques developed in the prior art, such as the method of cell aggregation by centrifugation commonly used in cell therapy.

The invention presently makes it possible to obtain cell structures comprising differentiated cells, in particular large cell structures, thereby making it possible to implant a restricted number of such structures at the level of the lesion and facilitating the manipulation of said structures by the surgeon. The invention also makes it possible to dispense with the use of any support matrix.

### SUMMARY OF THE INVENTION

The invention relates to a process for aggregation of cells, in particular of stem cells and/or of cells undergoing differentiation or cells in the process of differentiating, comprising:
a) bringing stem cells or cells undergoing differentiation into contact, in vitro or ex vivo, with magnetic particles, said bringing into contact making it possible to obtain magnetised cells, and
b) exposing said magnetised cells to a magnetic field, said exposure enabling the formation of a cell aggregate.

The process according to the invention enables optimal aggregation of the cells, typically stem cells, and promotes the organisation thereof and advantageously the differentiation thereof.

The invention also relates to the cell aggregate which can be obtained using such a process, said aggregate comprising magnetised cells, for example magnetised stem cells, magnetised stem cells in the process of differentiating, magnetised cells in the process of differentiating, and/or differentiated magnetised cells. It also relates to the use of one or more of these cell aggregates, or the result of the fusion of said aggregates, as tissue initiators which are injectable or implantable in vivo in animals.

Moreover, the invention relates to a process for preparing a tissue structure, comprising the implementation of a cell aggregation process according to the invention and, optionally, a step of culturing one or more cell aggregates obtained at the end of said aggregation process, and also to the tissue structure which can be obtained using such a process, said tissue structure comprising (i) magnetised cells, for example magnetised stem cells, magnetised stem cells in the process of differentiating, and/or magnetised cells in the process of differentiating, and also optionally differentiated magnetised cells, said cells being mature or immature, and identical or different, and (ii) an extracellular matrix. The invention also relates to the use of this tissue structure as a tissue substitute, it being possible for such a substitute to be implanted in vivo.

The invention makes it possible in particular to remove the technical obstacle of the limited size of the tissue initiators since it makes it possible to obtain aggregates comprising 10 times more cells than the aggregates obtained using the existing techniques, and therefore implantable tissue initiators of modulable size, in particular of large sizes, which can be used clinically. This increase in size of the differentiated aggregates probably results from the initial geometry obtained by virtue of the aggregation process according to the invention which is favourable to exchanges between said aggregate and the surrounding medium. In addition to being viable, the resulting cell aggregates do not, contrary to the existing cell aggregates, lose their long-term fusion properties. They can thus fuse with one another throughout the chondrogenesis process. The cell aggregates can also advantageously be cultured in large number in a single bioreactor, unlike the existing small aggregates which require the use of one tube per aggregate.

The present invention also makes possible the organisation of the cell aggregates in space so as to obtain any shape, in particular that of the lesion site, and the coculture of several organised cell types, for example of cartilaginous cells and of bone cells.

The process described, applicable on a large scale, also makes it possible to obtain a functional tissue without needing to combine it with a support matrix (always problematic with regard to questions of biocompatibility and degradation) and makes it possible to partly or even totally dispense with the use of expensive growth factors. The tissue substitutes, like the cell aggregates, obtained in the context of the present invention can in fact advantageously be implanted early, i.e. before the end of the differentiation process.

Moreover, by virtue of the incorporation of magnetic particles in the tissue structure, the process according to the invention enables the non-invasive monitoring of the in vivo development of this structure after its implantation, for example using the magnetic resonance imaging (MRI) technique well known to those skilled in the art.

### BRIEF DESCRIPTION OF THE FIGURES

FIG. 1: Magnetic devices

FIG. 1 shows examples of devices which can be used for the magnetic condensation of cells in the context of the present invention. From left to right: hexagonal network of ends of truncated magnetic needles bringing about, for example, the formation of aggregates of 250,000 cells above each end; soft-iron cross, magnetised by a permanent magnet, bringing about the formation of an aggregate of 4 million cells in the form of a cross; ring-shaped permanent magnet, bringing about the formation of a cell aggregate of 4 million cells of the same geometry.

FIG. 2: 3D bioreactor

FIG. 2 gives an example of bulk culture of cell aggregates. The geometry used is in this case a square network of truncated needle ends (FIG. 2B), magnetised by virtue of a permanent magnet placed below this network (FIG. 2A). In this example, 16 aggregates, each of 250,000 cells, can be cultured simultaneously (FIG. 2C).

FIG. 3: Magnetic condensation for obtaining a large cartilage initiator

This figure illustrates the advantage of the magnetic condensation for obtaining cartilaginous tissue by differentiation of stem cells.

FIG. 3A: an initial deposit of one million or several million cells according to a line or cross geometry results in the formation of spherical aggregates. During this geometry transition, the cell aggregate maintains a large area of exchange with the extracellular medium, favourable to the diffusion of nutrients and of oxygen to the heart of the aggregate (arrows pointing towards the vacant spaces).

FIG. 3B: The final volume of the aggregates obtained by means of this technique is much greater than that of the aggregates obtained by means of the conventional technique of confinement by centrifugation.

FIG. 3C: The differentiation of the stem cells can be evaluated by quantification of the expression of the gene encoding collagen II, related to that of the gene encoding collagen I. On this graph, it is observed that the increase in the number of cells confined by centrifugation from 250,000 to 1 million results in a drastic decrease in the expression of the genes of the cartilaginous matrix (comparison of the control with (1)). Conversely, when one million cells are confined by magnetic condensation, the expression of these same genes remains comparable with that in the case of a confinement by centrifugation of 250,000 cells (comparison of the control with (2)) and is more than 100 times greater than that in the case of a confinement by centrifugation of one million cells (comparison (1) and (2)).

FIG. 3D: The histological analysis of the tissue obtained from the magnetic line (one million cells on one centimetre) shows positive staining of the cartilaginous matrix (dashed circle).

FIG. 4: Assembly of cartilage initiators by fusion

FIG. 4A: The aggregates formed by magnetic condensation have an intrinsic capacity to fuse, as shown by the formation of “tissue bridges” (arrows) after simply bringing these aggregates into contact.

FIG. 4B: This property proves to be advantageous in a “bottom up” approach in which it is sought to form tissue substitutes for cartilage that are of suitable size and geometry for the patient's defect. Aggregates can be assembled by fusion so as to form cartilage “sticks”, which are themselves assembled into sheets before these sheets fuse to give thick cartilage.

FIG. 4C: This image shows the fusion of 16 units of 200,000 cells, 4 aggregates among the initial 16 being denoted by the arrows.

FIG. 4D: The histological analysis of an aggregate (stick of cartilage) obtained by fusion of two units reveals positive staining of the cartilaginous matrix (dashed circles).

FIG. 4E: The sequential fusion of sixteen units makes it possible to obtain a large continuous tissue (sheet). Moreover, the histological analysis of the tissue formed reveals positive staining of the cartilaginous matrix (area delimited by the dashes).

FIG. 5: Visualisation of a cartilage aggregate obtained by magnetic condensation

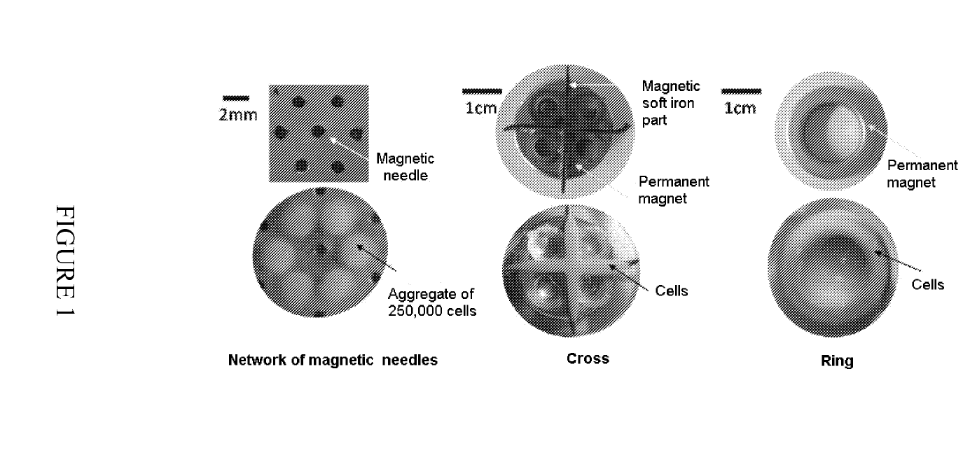

The presence of magnetic nanoparticles in the aggregate makes it visible by non-invasive volumetric MRI imaging. The images were taken with a Brucker 4.7T MRI instrument, using a 3D gradient-echo sequence (magnetic susceptibility-weighted) with TR/TE=20/5 ms, flip angle=25°, spatial resolution 50×50×50 μm. The cartilage aggregates observed were formed by magnetic condensation from 250,000 stem cells rendered magnetic (A: labelling [Fe]=0.05 mM, 30 min, 1 pg of iron per cell and B: labelling [Fe]=0.1 mM, 30 min, 2 pg of iron per cell) with magnetic nanoparticles, and placed in culture for 28 days.

### DETAILED DESCRIPTION OF THE INVENTION

The inventors have developed a process which very significantly improves the organisation of cells in the context of a cell aggregation process very advantageously capable of promoting the differentiation of cells, in particular cells undergoing differentiation and/or stem cells, typically mesenchymal stem cells (MSCs), embryonic stem (ES) cells or reprogrammed cells (induced Pluripotent Stem Cells or iPSCs, also identified in the context of the present invention by the expression “induced progenitor cells”), by applying to said cells the “cell aggregation by magnetisation” technique. This technique comprises (i) the introduction of magnetic particles into the cells (by spontaneous internalisation), then (ii) the formation and the organisation of cell aggregates having controlled geometric shapes via exposure of the magnetised cells to an external magnetic field. Step (i) of this process can be carried out in vitro or ex vivo. Step (ii) can be carried out in vitro, ex vivo or in vivo, according to the preference of those skilled in the art, preferably under conditions suitable for the intended subsequent application of the cell aggregate, for example preferably under conditions suitable for the stage of the differentiation process on which the cells are and/or for the stage of cell differentiation that it is desired to attain. Such a process offers clinical perspectives that were not previously envisagable given the technical constraints, previously explained, associated with the existing methods.

The invention thus relates to a process for cell aggregation, comprising:

a) bringing cells, in particular stem cells or cells undergoing differentiation, into contact, in vitro or ex vivo, with magnetic particles, said bringing into contact making it possible to obtain magnetised cells, and
b) exposing said magnetised cells to a magnetic field, preferably a focused magnetic field, said exposure enabling the formation of a cell aggregate.

The aggregation method according to the invention applies to all cell types which can be rendered magnetic and which are preferably capable of following a pathway of differentiation. The cells capable of being used are typically animal cells, preferably mammalian cells, even more preferentially cells of human beings. These cells can be chosen, for example, from stem cells, cells undergoing differentiation, or reprogrammed cells, typically induced progenitor cells. They may be cells of embryonic, foetal, neonatal and/or adult origin, and also mature or immature cells. They may, for example, be cells originating from blood, from bone marrow, from the placenta, from bone, from cartilage, from muscle, from skin or from adipose tissue. Examples of cells that can be used in the context of the invention comprise (without being restricted thereto): adult stem cells of mesenchymal origin (typically originating from the bone marrow or from the adipose tissue), embryonic stem cells, induced progenitor cells, peripheral blood progenitor cells, or progenitor cells from umbilical cord blood or from the musculoskeletal system (for example, myogenic precursor cells or mesenchymal stem cells).

The stem cells which can be used as cell therapy tools in the context of the invention are of embryonic origin [stem cells (SCs) which are embryonic stem cells (ESCs)] or non-embryonic origin (foetal, neonatal and adult tissues), and originate from an animal, typically from a mammal, preferably from a human being.

Embryonic stem cells (ESCs) have, in vitro, an infinite multiplication potential and can give rise to all the cell types. Such cells can be obtained without destruction of the embryo from which they are derived, for example using the technique described by Chung et al. (cf. Cell Stem Cell. 2008 Feb. 7; 2(2): 113-7). The use of such stem cells is particularly recommended for obtaining cardiomyocytes.

Non-embryonic stem cells may be mesenchymal stem cells (MSCs), induced progenitor cells, neuronal stem cells, endothelial progenitors, etc. They are, for example, derived from the synovium, from the periosteum, from bone marrow, from adipose tissue, from umbilical cord blood, from the placenta, from Wharton's jelly, etc. Preferred non-embryonic stem cells, which can be used in the context of the invention, are chosen from adult stem cells of mesenchymal origin or MSCs, and stromal cells (in particular for obtaining chondrocytes, hepatocytes or cardiomyocytes).

Certain types of non-embryonic stem cells have the advantage that they can be easily sampled and transplanted into the same individual (autografted), thus eliminating any risk of rejection. Thus, MSCs can, for example, be sampled by bone marrow puncture in the patient and then isolated by spontaneous attachment to a plastic support, before being used in the context of the present invention. Other sources of MSCs are, for example, adipose tissue, the synovium and the periosteum.

The genetic reprogramming of adult differentiated cells (cf. Yu J, Vodyanik M A, et al. (2007). “Induced Pluripotent Stem Cell Lines Derived from Human Somatic Cells”. Science 318 (5858): 1917-1920; Takahashi K, et al. (2007). “Induction of Pluripotent Stem Cells from Adult Human Fibroblasts by Defined Factors”. Cell 131 (5): 861-872) also makes it possible to obtain pluripotent cells with the same multiplication and differentiation capacities as embryonic stem cells. These cells, classified in the context of the present invention in the stem cell category, are called iPSCs (induced Pluripotent Stem Cells) or induced progenitor cells and can also, as previously indicated, be used in the context of the present invention.

The bringing of cells into contact, in vitro or ex vivo, with magnetic particles makes it possible to obtain “magnetised” cells according to the “magnetic labelling” or “magnetisation” technique. This step, conventionally carried out by incubating the cells with the magnetic particles, is a required step in the implementation of the process claimed and precedes the “magnetic aggregation” step which involves exposing the cells, the aggregation of which is desired, to a magnetic field.

The magnetisation of the cells is based on the binding of magnetic particles to the surface of the cells to be magnetised or on the internalisation of magnetic particles by said cells. The mechanism of internalisation of the nanoparticles by the cells is based on the invagination of the cell membrane followed by the formation of endocytotic vesicles filled with magnetic particles. These vesicles transport their content within the cytoplasm so as to deliver it to the late endosomes or lysosomes.

An example of a process for magnetisation of cells by internalisation of magnetic nanoparticles is described in the publication Wilhelm C. and Gazeau F. (Biomaterials. 2008; August 29(22):3161-3174). In this case, the incubation step is then followed by a “chase” period in cell culture medium without nanoparticles so as to allow complete internalisation of all the nanoparticles initially adsorbed at the surface of the cells. It is observed that, one to two hours after the beginning of the incubation, the particles are thus concentrated inside the cells, and their number ranges between approximately 10⁵ and approximately 10⁸ particles per cell, for example 10⁷ particles per cell (which corresponds to approximately 10 pg of iron).

Magnetic particles that can be used in the context of the present invention are magnetic microparticles or magnetic nanoparticles, preferably magnetic nanoparticles.

The size (average diameter) of the nanoparticles is less than 1 μm, typically between 5 nm and 999 nm, preferably between 5 and 250 nm, particularly preferably between 5 and 50 nm, even more preferentially between 5 and 15 nm, and is for example 6, 7, 8, 9 or 10 nm. Small particles (the size of which is typically less than 50 nm) will more readily have a tendency to internalise into the cells than large particles.

The size (average diameter) of the microparticles is greater than or equal to 1 μm, typically between 1 μm and 4 μm and preferably between 1 μm and 2 μm.

The particles that can be used in the context of the present invention are magnetic particles, i.e. particles consisting of at least one magnetic material, particles comprising several magnetic materials, and particles comprising at least one magnetic material optionally combined with other non-magnetic materials (hybrid particles). Examples of hybrid particles that can be used are nanoparticles of core-shell type, asymmetric particles (of Janus type), or porous or mesoporous structures (for example in combination with silica).

The magnetic material may be hard or soft, i.e. having strong or weak magnetic anisotropy.

Examples of magnetic materials that can be used in the context of the present invention are ferromagnetic, ferrimagnetic, superparamagnetic, antiferromagnetic or optionally paramagnetic materials.

A ferrimagnetic material that can be used in the context of the present invention may be, for example, a ferrite of spinel structure, for example chosen from maghemite, magnetite, nickel ferrite, cobalt ferrite, etc.

A paramagnetic material that can be used in the context of the present invention may be chosen, for example, from a gadolinium-doped material, a gadolinium oxide or a manganese oxide.

An example of a particle consisting of superparamagnetic material that can be used in the context of the present invention may be a ferrite (iron oxide, maghemite for example) nanoparticle, the size of which is preferably less than 20 nm.

The use of ferromagnetic or ferrimagnetic materials is preferred. Particularly preferred examples of ferro- or ferrimagnetic materials are iron oxides such as magnetite (Fe₃O₄) or maghemite (γFe₂O₃).

The material chosen may also consist of metal oxide (iron oxide, cobalt oxide, nickel oxide, manganese oxide, platinum oxide), of pure metals (for example iron, cobalt, nickel) or of metal alloys (for example CoPt, FePt), optionally in combination with non-magnetic metals (for example silver or gold).

The magnetic particles used in the context of the present invention can be in a crude form, can be bonded to one or more ligands (citrate for example) or polymers (dextran or polyethylene glycol (PEG) for example), can be partially or totally covered or coated with such ligands or with such polymers, or else can be encapsulated, for example in micelles, carbon or titanium nanotubes, liposomes, cell vesicles or viral vectors, using techniques well known to those skilled in the art.

Particle doses which are effective in the context of the present invention, and which can be readily determined by those skilled in the art, are for example approximately 10 pg of iron per cell (i.e. approximately 0.1 mg of iron for approximately 10 million cells). Such doses are approximately one thousand times lower than those used in imaging (20-50 μmol/kg, i.e. approximately 0.1 g of iron for one patient). By way of comparison, the total weight of iron in the human body is about 3-4 g, while 1 to 2 mg are renewed each day.

The studies carried out to date on the iron oxide nanoparticles conventionally used in the context of the magnetisation technique have not revealed any toxic nature. Nanoparticles consisting of iron oxide have, moreover, been approved as contrast agents that can be used in magnetic resonance imaging (MRI) for clinical use.

In the context of their experiments, the inventors have systematically performed cytotoxicity and functionality tests on the particles used. They have thus been able to demonstrate the innocuousness of the particles, in particular on cell proliferation and differentiation. The use of such particles thus proves to be particularly advantageous in animals, in particular mammals, and preferably human beings.

Those skilled in the art are, moreover, able to control, by means of the conditions for carrying out the methods described in the context of the present invention, the amounts of nanoparticles internalised by the cells, in particular by the stem cells, and therefore to apply an external magnetic field of modulable intensity, which can be controlled on a cellular scale.

The incubation, for example for 30 minutes, for example of human mesenchymal stem cells, with a medium containing iron oxide nanoparticles (approximately 8 nm in diameter) at iron concentrations ranging from 0.05 mM to 5 mM results in intracellular iron weights of approximately 1 to 31 pg per cell. An incubation of only 1 minute is also possible. This time is in fact sufficient to enable the adsorption of the nanoparticles onto the cell membrane. In the absence of a chase period, these nanoparticles will however localise essentially at the surface of the cell membrane.

The formation of dense cell aggregates from the magnetic cells is obtained in the context of the present invention using a magnetic field, which is advantageously focused, or of several magnetic fields, which are preferably focused and/or characterised by a sizable magnetic-field gradient (strong cellular magnetic force for example of between approximately 0.1 nN and approximately 10 nN).

These magnetic fields are typically generated by magnets or by magnetisable parts. Strong magnetic fields can in particular be generated, for example, by permanent magnets (hard magnetic materials consisting, for example, of neodymium, nickel, iron, etc.), by parts magnetised by a permanent magnet placed close by, or by electromagnets (parts made of copper or of gold for example).

These magnets can be machined to the dimensions and to the geometry desired. Each time, several of these magnets can be combined to create a greater variety of shapes or patterns.

The conventional microfabrication techniques known to those skilled in the art can be used to deposit, in a controlled manner, onto a substrate, (i) for example a ferromagnetic material so as to produce a permanent magnet or (ii) a conductive material (typically a metal such as gold or copper) so as to produce an electromagnet. In the latter embodiment, the magnetic properties then depend on the passing of an electric current in the conductive material. Controlling the strength of the electric current over time makes it possible to modulate the strength of the magnetic force applied.

Step b) of the aggregation process according to the invention, which corresponds to the exposure of the magnetised cells to a magnetic field, thus typically consists of the exposure of said cells to one or more magnetic shapes.

Said shape can be chosen from a spot, the tip or the end of a truncated needle, a line, a cross, a star, a ring, a disc, a circle, or several of one and/or other of these shapes, for example a collection of spots or a collection of lines, preferably organised in a network (for example a network of ends of truncated needles as a set of spots, or a mesh as a set of lines). Said shape is advantageously chosen from a network of ends of truncated needles, one or more lines, a cross and one or more rings, for example arranged concentrically.

In one particular embodiment, the tip of a needle or the end of a truncated needle (for example approximately 1 mm in diameter), consisting of a suitable magnetic material as previously described or magnetised by a permanent magnet, can be used to create aggregates having the shape of a hemisphere. Several of these truncated needle ends, which are then “isolated” magnetic sources, can be combined in order to obtain several aggregates on the same support or substrate. The organisation of the cell aggregates is then defined by that of the magnetic needle tips or of the truncated magnetic needle ends.

The assembling or the combining of these needle ends may reflect, as previously explained, a regular organisation (for example a square or hexagonal network, a spiral, etc.) or an irregular organisation (reflecting, for example, the geometry of the patient's defect). The spacing of the magnets can in fact be easily modulated by those skilled in the art.

The spacing chosen between the magnets makes it possible to control the fusion of the cell aggregates obtained by inducing a more or less close contact between the latter. Thus, for example, in the case of a network of truncated magnetic needles, the spacing between the ends of the needles is, for example, between 1 and 20 mm. This spacing is preferably at least 1 mm, for example 2, 3, 4, 5, 10 or 15 mm.

In another particular embodiment, magnets or magnetisable parts of linear geometry are used. Parts, for example, made of soft iron, can be used to form “lines” of cells of modulable width and length. A typical length is, for example, between 0.5 cm and 2 cm, or between 1 cm and 2 cm, for a thickness of between 0.1 mm and 1 mm, typically of approximately 0.5 mm, for example of 0.2, 0.3, 0.4, 0.6, 0.7, 0.8 or 0.9 mm. As for the isolated magnets, several of these magnetic lines can be combined to form, for example, crosses, stars, meshes or combinations of lines spaced out to a lesser or greater extent (between 0.5 mm and 2 cm, typically between 1 mm and 2 mm, preferably between 1 mm and 1.5 mm, for example 1.1, 1.2, 1.3 or 1.4 mm).

In yet another particular embodiment, the use of a ring-shaped magnet makes it possible to obtain a cell aggregate of the same geometry. Several of these rings can be assembled as previously explained, for example concentrically.

The process according to the invention thus makes it possible to create three-dimensional (3D) patterns of which the geometry and the spatial organisation are modulable and can be completely determined (isolated or branched cell aggregates, cell aggregates in the form of lines or in the form of a square network, cross-shaped cell aggregates, cell aggregates in the form of a hexagonal network, etc.). The linear geometries obtained by magnetic condensation are particularly effective in that they make it possible to increase the area of exchange with the ambient medium and thus promote nutrient and oxygen diffusion.

The cell aggregation density can be easily controlled by those skilled in the art by varying the strength of the magnetic force applied, which can be between 1 and 5000 piconewtons (pN), for example between 1 and approximately 1000 pN, preferably between approximately 100 and approximately 900 pN, and preferably approximately 200, 300, 400, 500, 600, 700 or 800 pN. These force strengths have in particular been tested for stem cells intended for differentiation into chondrocytes, but also for other cell types, such as monocytes, macrophages, endothelial progenitors, endothelial cells, lymphocytes and mesenchymal stem cells.

The aggregation method according to the invention can be carried out on any type of support or substrate, from those which interact weakly with the cells, such as glass, to those which interact strongly by facilitating cell adhesion.

The interactions with the cells can also be generated or promoted by the nature of the chosen constituent material of said substrate or by a prior preparation or treatment of said material, for example the partial or total coating thereof, using a substance which promotes cell adhesion. Such a substance can be chosen, for example, from gelatin, fibronectin, an RGD sequence, and cells.

The substrate can also or otherwise be “pretreated” by incubation for example in serum.

The support or substrate on which a combination or a network of magnets or of magnetisable parts can be organised is preferably a two-dimensional substrate, typically a surface consisting of a material chosen, for example, from glass, plastics, commercial plastics treated for cell culture, organic and inorganic polymers, and mineralised matrices.

This surface is preferably flat. It is, for example, a culture dish, for example a culture dish not pretreated using a substance which promotes cell adhesion.

In the particular case of the tissue engineering of implants or tissue substitutes which are “two-phase” (i.e. comprising two cell types, such as bone cells and cartilaginous cells), this substrate could correspond to one or more sheets of osteocytes or to an osteoinductive matrix, for instance a matrix incorporating calcium phosphate or hydroxyapatite.

The aggregation process according to the invention can also be carried out in the presence of a support matrix (three-dimensional substrate, 3D substrate or three-dimensional cell adhesion support which is exogenous). The three-dimensional support matrices conventionally used are generally selected from a porous gel, for example a hydrogel or a polysaccharide-based gel; a synthetic polymer; a partially mineralised matrix; a biological polymer such as collagen; and bone, optionally decellularised bone. In this case, the cell aggregates preferably form not at the surface of the support matrix, but in situ, i.e. in said support matrix. The process according to the invention is, however, advantageously and preferably carried out in the absence of such a 3D support matrix, thus limiting the risks of incompatibility in the case of implantation in animals.

The substrate, typically the two-dimensional substrate, is advantageously placed, in the context of the aggregation process according to the invention, close to, for example above or below, the magnetic source (magnet or magnetisable part combined with a permanent magnet). The magnetised cells are then deposited close to the magnetic source, typically in the form of a dilute suspension, on all or part of the substrate, preferably on the whole of the substrate, or in the form of a concentrated suspension on one or more parts of the substrate. Of course, the magnetised cells can, according to another embodiment of the invention, be deposited on the substrate or close to the substrate before exposure of the latter to the magnetic source.

The number of cells intended to form an aggregate which are used ranges between approximately 0.1 and approximately 4 million cells, for example between approximately 300,000 cells and 3 million cells, typically 400,000, 500,000, 600,000, 700,000, 800,000 or 900,000 cells, and several of these aggregates may be subsequently combined. The number of cells used is, for example, 0.2, 0.5, 1 and 2 million cells, preferably 1 million cells.

Whether the cells are deposited in the form of a dilute or concentrated suspension, the virtually instantaneous creation of cell-dense areas and cell-sparse areas can be observed the moment they are exposed to the magnetic field(s).

The duration of exposure of the stem cells to the magnetic field(s) in order to create an aggregate having a cell density which is in particular clinically exploitable is between approximately 1 minute and several tens of days depending on the nature of the cells used, the desired degree of cell differentiation and/or the desired shape of the aggregate. For example, in the case of chondrogenesis, this time can be between 1 minute and the complete duration of the differentiation cycle (28 days), advantageously between 1 minute and 1 hour, preferably between 20 min and 1 hour.

The exposure of the stem cells to the magnetic field(s) for the selected duration can be continuous or transient. It can, for example, be transient in particular for the first 10 to 20 minutes of implementation of step b) of the process according to the invention, when said step lasts more than one hour.

Differentiation factors (growth factors) are expensive elements which are essential in the prior art processes currently used, for example for stimulating chondrogenesis in vitro. They promote, among other things, the aggregation of cells when said aggregation is necessary for the maturation and differentiation process. However, the aggregation process according to the invention makes it possible to partially or even totally dispense with the use of such factors, for example, typically TGFβ1, TGFβ3 and/or BMP-2.

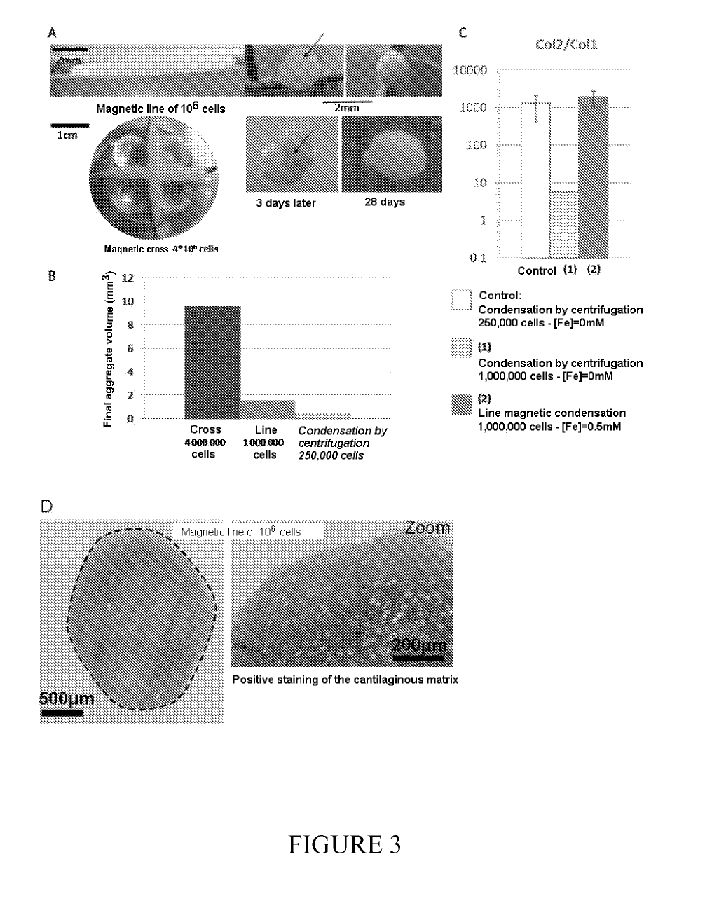

The process according to the invention makes it possible to create, by magnetic condensation, dense cell assemblies which are capable of differentiation, which have high fusion capacities sustained over time and which exhibit a high in vivo integration potential. The differentiated aggregates obtained (for example the cartilaginous tissue initiators) by means of the invention are much larger in size than those obtained using the known techniques. Thus, the final volume of the differentiated cell aggregate obtained according to the invention is multiplied approximately 10-fold compared with the volume of an aggregate obtained using the known techniques. For example, for approximately 4 million cells used, the final volume of the cell aggregate obtained using an aggregation method according to the invention can thus reach or even exceed 10 mm³, compared with approximately 1 mm³ under the prior art conditions. Unexpectedly, the inventors have in fact noted that the initial spread-out geometry is not maintained and spontaneously and systematically develops towards a spherical geometry (see FIG. 3).

The present invention also relates to the cell aggregates, typically the spherical cell aggregates, which can be obtained using a process as previously described, consisting (i) of cells, preferably of stem cells, and optionally and advantageously also of differentiated cells, and/or of cells (for example stem cells) undergoing differentiation, and also optionally and advantageously (ii) of an extracellular matrix (secreted by the differentiated cells), advantageously devoid of necrotic core. Typically, the cells present in the aggregate are or comprise magnetised cells.

The shape and the size of the aggregate obtained can be adjusted by those skilled in the art, as previously explained, by varying the nature and/or the shape or geometry of the magnetic source used, and/or the number of magnetised cells exposed to a magnetic field.

The cell density of the aggregate can be adjusted by those skilled in the art by varying the number of cells forming the aggregate and/or the strength of the magnetic force exerted on the cells. This strength can itself be easily modulated by those skilled in the art able to select, according to their needs, the nature and the amount of magnetic material combined with the cells, the nature and the shape of the magnetic source, and also the distance between the cells and said source. The strength can, for example, be modulated as previously explained by varying the experimental conditions of the magnetic labelling.

The present invention also relates to the use of the cell aggregates presently described, as tissue initiators which are injectable (in suspension) or implantable in vivo in animals, in particular mammals, preferably humans. The maximum size of the aggregates which can be injected in suspension depends on the diameter of the needle used, which is itself linked to the injection site.

These cell aggregates can also and advantageously be assembled (fusion) into cell structures of controlled shapes and large sizes. It is also possible, by virtue of the present invention, to bring about the fusion of cell aggregates that are different in that they consist of different cell types (bone and cartilaginous cells, for example).

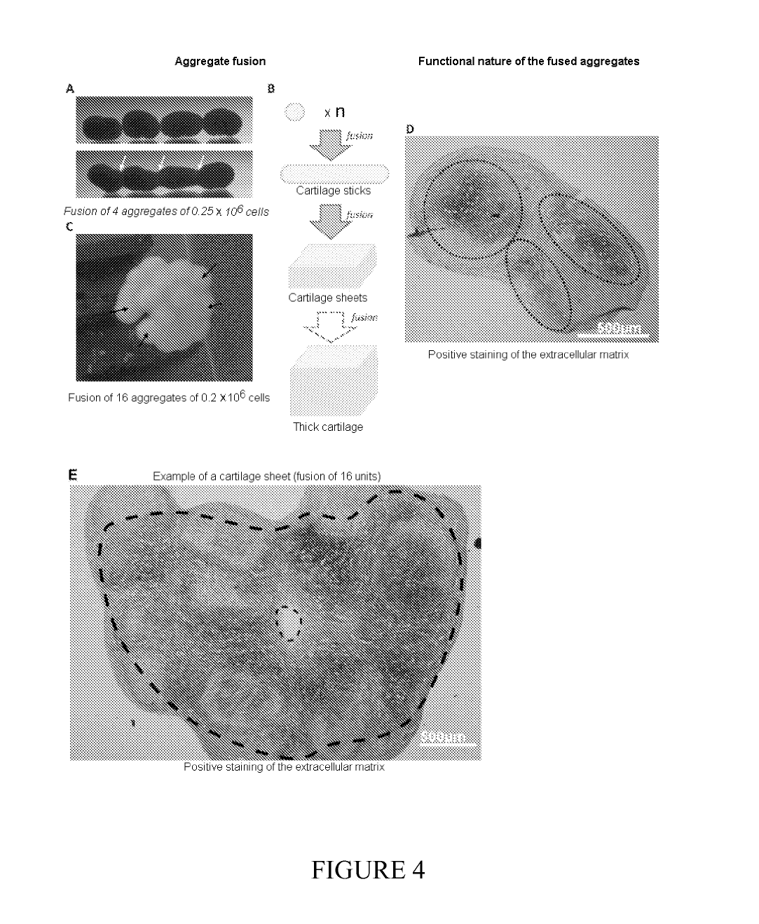

The present invention thus also relates to a process for preparing a tissue structure, comprising the implementation of a cell aggregation process according to the invention. The inventors have in particular demonstrated, as previously explained, that the process according to the invention, termed magnetic condensation process, makes it possible to dispense with the cell centrifugation step for obtaining cell aggregates of modulable sizes and shapes (or geometry), preferably of large sizes, after exposure of said cells to a magnetic field. It is thus possible to obtain rods (see FIG. 4A for example) or else cell/tissue sheets, which can themselves be assembled (owing to their great capacity to fuse), in order to i) increase the thickness of a tissue structure or ii) assemble structures comprising different cell types (for example bone or cartilage, cartilage and stem cells, or else bone and stem cells).

The process according to the invention for preparing a tissue structure can comprise a step of culturing a cell aggregate according to the invention, or of coculturing several of these cell aggregates (consisting of identical or different cells).

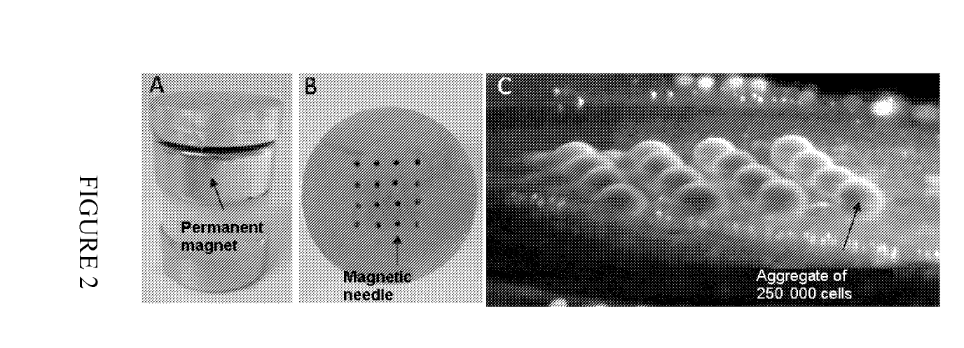

The aggregates obtained after exposure to a magnetic field created by isolated micromagnets can be of size similar to that of the aggregates obtained using the process of aggregation by centrifugation (termed condensation by centrifugation) (see FIG. 2). The advantageous contribution of the present process, even for aggregates of similar sizes, is to make possible the simultaneous cell differentiation to the desired stage of a very large number of aggregates in the same bioreactor (see FIG. 2C), whereas the technique involving centrifugation imposes the use of a single aggregate per centrifugation tube. The aggregation process according to the invention is thus possible and advantageously carried out without a centrifugation step.

Moreover, several of these aggregates can fuse to give rise to larger structures.

The culturing step of the process according to the invention can thus involve one cell aggregate or simultaneously several cell aggregates cultured in an incubator for a period of time which can be between one hour and several days, or even several months. Thus, in the case of chondrogenesis, the culturing step can last between one day and 28 days. This culturing step is generally carried out in the presence of growth factors and/or of physical stimulae. Thus, for example, the incubation of the cell aggregates in a chondrogenic medium containing one or more growth factors such as those previously mentioned, preferably TGFβ3, is desirable if it is desired to obtain a cartilaginous tissue structure.

The similar behaviour, during the first days of the differentiation process, of, on the one hand, aggregates cultured in the presence of one or more chondrogenesis-inducing growth factors and of, on the other hand, aggregates cultured in the absence of such factors demonstrates that the process according to the invention for preparing a tissue structure can be carried out in the absence of growth factor, preferably in the absence of TGFβ1, TGFβ3 and/or BMP-2, at least during the aggregation step, and advantageously also during the first day or the first 2, 3, 4, 5, 6, 7 or 8 days of the culturing step.

In one particular embodiment of the present invention, the first aggregate culturing phase is carried out in the absence of a growth factor and is advantageously followed by a second culturing phase in the presence of one and/or other of the growth factors normally used by those skilled in the art, depending on the cell type(s) concerned and the nature of the tissue structure(s) that it is desired to obtain.

The second culturing phase can also comprise or otherwise consist of the injection or the implantation of the aggregates having being subjected to the first culturing phase. In this case, the in vivo environment takes over from the growth factors introduced into the culture medium.

The aggregated cells may optionally be exposed, during all or part of the culturing step, for example 1, 3, 7, 14 or 28 days, to a magnetic field as previously defined, continuously or sequentially.

The phase for culturing, as described in the context of the present invention, the aggregated cells or the aggregates obtained can be carried out in the presence of a magnetic field as previously defined, continuously or sequentially. This magnetic field can be created using one or more magnetic sources, which sources can be moved closer to one another as explained above in the present description in order to promote the fusion of the aggregates so as to form a larger tissue structure of desired shape.

Just like the aggregation process, the process according to the invention for preparing a tissue structure can be advantageously carried out in the absence of a three-dimensional (3D) support matrix.

The present invention relates, moreover, to the tissue structure obtained or capable of being obtained using a process as described in the present document.

Such a structure comprises (i) differentiated cells, for example cartilaginous cells, typically differentiated magnetised cells, and (ii) an extracellular matrix, for example a cartilaginous extracellular matrix (composed of collagen type II fibres and/or of proteoglycans such as aggregan) if the differentiated cells present are cartilaginous cells. It can also comprise stem cells and/or cells in the process of differentiating. These cells are typically magnetised cells.

The present invention also relates to the advantageous use of such a tissue structure according to the invention, as a tissue substitute. Such a substitute can be implanted in vivo in animals, in particular mammals, preferably humans.

The tissue structure can, as previously explained, be in the form of a tissue rod, sheet, patch or stick or in any other form of interest.

Such a tissue structure can also advantageously be used in research in vivo, ex vivo or in vitro.

The present invention also relates to a method for monitoring the development in vivo of such a tissue structure.

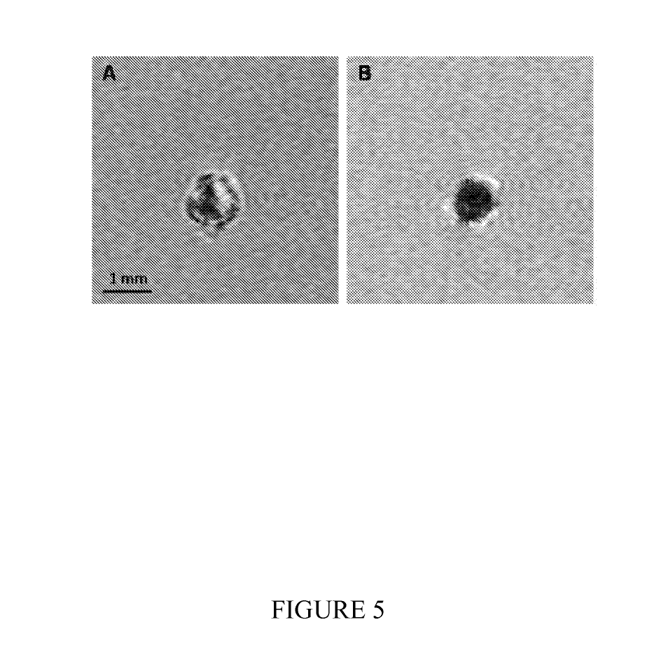

The cell aggregates and cell or tissue structures according to the invention comprise, as previously explained, magnetised cells such that their visualisation and localisation is possible by imaging, for example using an MRI instrument (FIG. 5).

The present invention makes it possible, for the first time and very advantageously, to control the differentiation and also the outcome of cells grafted into host tissues.

### EXAMPLES

### Example 1

### Aggregation Method Applied to Obtaining Chondrocytes from Human Mesenchymal Stem Cells (hMSCs)—Reference Conditions

### Material and Method

Aggregate Formation

The human mesenchymal stem cells (hMSCs) used come from commercial aliquots (Lonza). These cells are incubated for 30 min with a suspension of iron oxide (maghemite) nanoparticles at an iron concentration of 0.1 mM. After an overnight chase in a nanoparticle-free culture medium, 250,000, 500,000 or 1,000,000 cells are deposited on a glass substrate (100 μm thick) placed above a magnetic needle. This process results in the formation of spherically-shaped cell aggregates. The magnetic force exerted on the cells of the aggregate ranges from 70 to 400 pN.

| | | | | Number |
|---|---|---|---|---|
| Condition | Labelling | Substrate | Magnetic force | of cells in the |
| No. | [Fe] = | thickness | applied to the cells | aggregate |
| 1.1 | 0.1 mM | 100 μm | 70 to 400 pN | 0.25 × 10⁶ |
| 1.2 | 0.1 mM | 100 μm | 70 to 400 pN | 0.50 × 10⁶ |
| 1.3 | 0.1 mM | 100 μm | 70 to 400 pN | 1.00 × 10⁶ |

Cell Differentiation

In the case of cartilaginous tissue engineering, the mesenchymal stem cell aggregates are advantageously cultured in an incubator (37° C., 5% CO₂) for 28 days in a chondrogenic induction medium containing in particular the growth factor TGFβ3. This medium is renewed twice a week.

Analysis of the Tissue Formed

After the 28 days of culture in a chondrogenic medium, the aggregates were analysed by histology and by quantitative PCR in order to demonstrate the presence of cartilaginous matrix. This matrix is composed of collagen type II fibres and proteoglycans, which associate a protein core with glycosaminoglycan chains. An example of proteoglycans found in abundance in cartilage is aggregan.

The quantitative PCR makes it possible to evaluate the expression of the genes of the two major components of the matrix, collagen II and aggregan. Moreover, the ratio of the expression levels of the genes encoding collagen II and collagen I gives an indication of the progression along the chondrogenic differentiation pathway: mesenchymal cells, which are undifferentiated, synthesise predominantly collagen I, while chondrocytes produce collagen II in abundance.

The role of the histological stainings with toluidine blue and with safranin-O is to reveal the presence of glycosaminoglycans through a metachromatism from blue to violet or an orange coloration, respectively.

### Histology

The samples are first of all fixed in formalin (12 h of incubation at 4° C.) before being frozen in an OCT (Optimal Cutting Temperature compound) matrix in a bath of liquid-nitrogen-cooled isopentane. Using a microtome cryostat, 6 μm sections are then cut and are subsequently deposited on pretreated slides. Before the actual staining, the sections are again fixed in 1% formalin for 10 minutes at ambient temperature, and then rinsed in a bath of tap water.

### Toluidine Blue Staining

Staining of the slides for 1 to 2 minutes with a 0.5% toluidine blue solution (500 mg in 20 ml of 95% ethanol and 80 ml of distilled water).
Rinsing in a bath of tap water.
Dehydration: three successive baths in 100% ethanol (2 min; 2 min; 5 min) and three successive baths in toluene (2 min; 2 min; 5 min).
Mounting of the slides with Eukit.

### Safranin-O Staining

Haematoxylin Staining
Staining for 3 minutes with a 0.2× solution diluted in distilled water.
Rinsing for 2 minutes in a bath of tap water.
Differentiation in a bath of acid alcohol (1% hydrochloric acid HCl in 70% ethanol) for 15 seconds.

Fast Green staining
Staining for 3 minutes with a 1:5000 aqueous solution (40 mg in 200 ml of water).
Rapid rinsing in a bath of 1% acetic acid.

Safranin-O Staining
Staining for a maximum of 3 minutes in an aqueous 0.1% safranin-O solution.
Rinsing in a bath of 95% ethanol.
Dehydration: three successive baths in 100% ethanol (2 min; 2 min; 5 min) and three successive baths in toluene (2 min; 2 min; 5 min).
Mounting of the slides with Eukit.

### Quantitative PCR (Polymerase Chain Reaction)

The total RNA contained in the cells after the differentiation phase is extracted with the Machery-Nagel kit according to the protocol indicated by the supplier. The RNA obtained is treated with DNAse in order to avoid any contamination with genomic DNA. The complementary DNA (cDNA) is then synthesised using the SuperScript II Reverse Transcriptase (Invitrogen) with a final concentration of 1 μg of total RNA for 100 μl. Real-time quantitative PCR is performed with the ABIPRISM 7900 apparatus sequence Detection System and SYBR Green dye (Applied-Biosystems), reference being made to the protocol indicated by the supplier. The PCR primers are constructed with the Primer Express program (Applied Biosystems). The mRNA transcription levels are standardised relative to the expression level of a reference gene: RPLP0 (acidic ribosomal phosphoprotein P0). The specificity of the transcript is confirmed by means of a melting curve formed at the end of the PCR. The established fluorescence detection threshold makes it possible to detect a threshold cycle (Ct) and to quantify the relative expression of a specific gene. The level of mRNA corresponding to the gene of interest (denoted R) is expressed relative to the level of the reference gene RPLP0: ΔCt=Ct_{R}−Ct_{RPLP0}. For each gene R, reference is made to a negative control sample having been cultured without differentiation medium. The amount of mRNA corresponding to the gene R is expressed relative to this negative control, and is equal to 2^{(-ΔΔCt)}, where ΔΔCt=ΔCtₛₐₘₚₗₑ−ΔCt_{negative control}.

### Sequence of the Primers Used:

The chondrogenesis of the hMSCs is measured by the expression level of three particular genes:
AGN: gene encoding aggregan, a cartilaginous matrix component;

Fwd:
(SEQ ID No. 1)
TCTACCGCTGCGAGGTGAT
Rev:
(SEQ ID No. 2)
TGTAATGGAACACGATGCCTTT

Col2A: gene encoding an α chain of collagen II, present in hyaline cartilage.

Fwd:
(SEQ ID No. 3)
ACTGGATTGACCCCAACCAA
Rev:
(SEQ ID No. 4)
TCCATGTTGCAGAAAACCTTCA

Col1A: gene encoding an α chain of collagen I, present in fibrous cartilage and synthesised by mesenchymal cells which have not yet differentiated.

Fwd:
(SEQ ID No. 5)
GCTACCCAACTTGCCTTCATG
(SEQ ID No. 6)
Rev:
TTCTTGCAGTGGTAGGTGATGTTC.

In order to evaluate the progression in the differentiation, the Col2A/Col1A expression ratio and the AGN expression are measured.

### Results

The aggregates of 250,000, 500,000 or 1,000,000 mesenchymal stem cells show, at the end of the 28 days of chondrogenesis, an expression of the genes characteristic of the cartilaginous matrix (AGN and Col2A/Col1A) that is comparable with that of the control, i.e. an aggregate of 250,000 cells formed by centrifugation (standard condition in the literature).

### Example 2

### Aggregation Method Applied to Obtaining Chondrocytes from Human Mesenchymal Stem Cells (hMSCs)—Variation in the Compactness of the Aggregates

### Material and Method

The variation in the compactness of the cell aggregates is obtained by increasing or decreasing the magnetic force applied to the cells during the aggregate formation with respect to the standard condition presented in Example 1. This force is modulated, either by means of a variation in the magnetic labelling of the cells, or by means of a deposit on a glass substrate which is thicker. The experimental conditions tested are reproduced in the following table.

| | | | | Number |
|---|---|---|---|---|
| Condition | Labelling | Substrate | Magnetic force | of cells in the |
| No. | [Fe] = | thickness | applied to the cells | aggregate |
| 2.1 | 0.5 mM | 100 μm | 140 to 900 pN | 0.25 × 10⁶ |
| 2.2 | 0.5 mM | 400 μm | 140 to 700 pN | 0.25 × 10⁶ |

The aggregates are formed as explained in Example 1 and cultured for 2 days in the chondrogenic induction medium including TGFβ3. During these two days, their contraction dynamics are monitored.

### Results

The aggregates have the same contraction dynamics.

### Example 3

### Aggregation Method Applied to Obtain Chondrocytes from Human Mesenchymal Stem Cells (hMSCs)—Deposit in “Spread Out” Geometry

### Material and Method

The initial geometry is modified with respect to Example 1 through the use of magnetisable soft iron parts machined in the form of lines or of a cross 0.5 μm thick. The experimental conditions used are reproduced in the table below. The depositing, aggregation, aggregate-culture and analysis methods are identical to those of Example 1.

| Condition | Labelling | Substrate | Magnetic force | Number of cells | | |
|---|---|---|---|---|---|---|
| No. | [Fe] = | thickness | applied to the cells | in the aggregate | Geometry | |
| 3.1 | 0.05 mM | 100 μm | 10 to 100 pN | 1.0 × 10⁶ | Line - 1 | cm |
| 3.2 | 0.1 mM | 100 μm | 20 to 240 pN | 1.0 × 10⁶ | Line - 1 | cm |
| 3.3 | 0.1 mM | 100 μm | 20 to 240 pN | 2.0 × 10⁶ | Line - 2 | cm |
| 3.4 | 0.5 mM | 100 μm | 50 to 450 pN | 1.0 × 10⁶ | Line - 1 | cm |
| 3.5 | 0.5 mM | 100 μm | 50 to 450 pN | 2.0 × 10⁶ | Line - 2 | cm |
| 3.6 | 2.0 mM | 100 μm | 100 to 1000 pN | 1.0 × 10⁶ | Line - 1 | cm |
| 3.7 | 0.1 mM | 100 μm | 20 to 240 pN | 4.0 × 10⁶ | Cross - 4 × 1 | cm |

### Results:

Condition 3.6 results in a matrix gene expression similar to that of the negative control (250,000 cells confined by centrifugation and cultured in chondrogenic medium without TGFβ3).
Condition 3.4 is more favourable to the formation of cartilaginous matrix than condition 3.5.
Conditions 3.1, 3.2, 3.3 and 3.4 result in cartilaginous matrix synthesis which is comparable with the positive control (250,000 cells confined by centrifugation and cultured in chondrogenic medium with TGFβ3).

### Example 4

### Fusion of Aggregates of Human Mesenchymal Stem Cells (hMSCs) in the Process of Undergoing Chondrogenesis—Application to the Formation of Tissue Substitutes of Controlled Form and Geometry

### Material and Method

Aggregates of human mesenchymal stem cells are formed by magnetic condensation and cultured in chondrogenic medium as explained in Example 1.

The type of magnet used to form the aggregates is either a magnetic needle (as in Example 1), or a magnetic line (as in Example 2). Several of these aggregates (for example 2, 4, 7, 16) are brought into contact at various times of the chondrogenesis process (for example day 0, 1, 4, 7, 10 or 16), which initiates their spontaneous fusion into a single cohesive aggregate. This bringing into contact may be spontaneous, when for example the aggregates are close to one another (conditions 4.1, 4.4, 4.6, 4.7). It may also be induced manually and maintained through the presence of an underlying magnet (magnetic needle or line depending on the condensation condition). Examples of induced fusion are provided by conditions: 4.2, 4.3, 4.5, 4.9, 4.10, 4.11, 4.12. For the fusion of a large number of units, a sequential fusion process can also be used. For example, in condition 4.12 concerning the fusion of 16 units in the end, 2 units are first of all fused on D7 (formation of 8 doublets). The doublets formed are then fused to give quadruplets on D11 (formation of 4 quadruplets). Finally, the four quadruplets are fused to give a sheet on D16. This sheet is the product of the sequential fusion of the initial 16 units.

The experimental conditions tested are summarised in the table below. The fusion time denotes the number of days having elapsed at the moment of fusion after the initiation of chondrogenesis.

| | Magnetic | Number of | Number of | | |
|---|---|---|---|---|---|
| Condition | condensation | cells per | fusing | Fusion | |
| No. | condition | aggregate | aggregates | time | Fusion type |
| 4.1 | 1.1 | 0.25 × 10⁶ | 2 | Day 1 | Spontaneous |
| | | | | | 2 mm spacing between |
| | | | | | the magnetic needles. |
| 4.2 | 1.1 | 0.25 × 10⁶ | 2 | Day 10 | Induced |
| 4.3 | 1.1 | 0.25 × 10⁶ | 2 | Day 16 | Induced |
| 4.4 | 1.1 | 0.25 × 10⁶ | 7 | Day 0 | Spontaneous |
| | | | | | 1.5 mm spacing between |
| | | | | | the magnetic needles. |
| 4.5 | 1.1* | 0.20 × 10⁶ | 16 | Day 10 | Induced |
| 4.6 | 3.2 | 1.0 × 10⁶ | 2 | Day 4 | Spontaneous |
| | | | | | 1.5 mm spacing between |
| | | | | | the magnetic lines. |
| 4.7 | 3.4 | 1.0 × 10⁶ | 2 | Day 4 | Spontaneous |
| | | | | | 1.5 mm spacing between |
| | | | | | the magnetic lines. |
| 4.9 | 1.1 | 0.25 × 10⁶ | 4 | Day 7 | Induced |
| 4.10 | 1.1 | 0.25 × 10⁶ | 4 | Day 9 | Induced |
| 4.11 | 1.1 | 0.25 × 10⁶ | 4 | Day 15 | Induced |
| 4.12 | 1.1 | 0.25 × 10⁶ | 16 | Day 8 | Induced |
| | | | | Day 11 | (sequential fusion) |
| | | | | Day 15 | |

*Compared with condition 1.1, the number of cells of 0.25 × 10⁶ cells per aggregate is replaced with 0.20 × 10⁶ cells per aggregate.

### Results

The proximity of the magnets inducing the formation of the cell aggregates results in a spontaneous and early fusion of these aggregates.
In the case of an early fusion (day 0, 1, 4), total fusion of the aggregates into a homogeneous single aggregate is observed.
In the case of a late fusion (day 7, 10, 16), fusion of the aggregates into an assembly which is cohesive, but the structure of which is marked by the presence of distinct units, is observed.
The time at which the aggregates are brought into contact can be completely controlled.
In the case of a late fusion, the size of the final aggregate is similar to the sum of the sizes of the units of which it is composed, whereas, in the case of an early fusion, additional compaction of the aggregate is observed.
Conditions 4.4 and 4.12 result in the formation of a tissue substitute which has an abundant cartilaginous matrix.
Conditions 4.1, 4.2, 4.3, 4.9, 4.10 and 4.11 result in the formation of tissue substitutes which have a cartilaginous matrix.
In all the conditions, a continuous tissue is formed.
Conditions 4.9, 4.10 and 4.11 result in expression levels of the collagen II and aggregan genes which are not significantly different.
The comparison of the collagen II expression level of the aggregates of 10⁶ cells, formed by magnetic fusion (conditions 4.9, 4.10, 4.11), with that of the aggregates also of 10⁶ cells, formed by centrifugation, shows that this expression level is significantly higher for the aggregates resulting from the magnetic fusion (3473±654) and for those formed by the customary centrifugation technique (880±291) (statistical test: Student's t test).

The presence of a chondrogenesis-inducing growth factor (TGFβ3, TGFβ1, BMP-2 or the like) or of a cocktail of factors appears to be useful only for a sustained culture of the aggregates (7-28 days). In this case, it is then desirable to create, in vitro, an environment favourable to the entire process, ranging from the cell condensation to the differentiation and the production of cartilaginous matrix.

In the context of an early implantation of the mesenchymal stem cell aggregates, the first step of cell condensation can be induced in vitro before implantation or injection of the initiators, or directly in vivo. The rest of the process (differentiation, matrix production) can be stimulated by the factors secreted in vivo by the native cells. Such an early implantation may take place after 2 or 3 days of culture in vitro, and preferably after one day of culture.

### Example 5

### Culturing of Mouse Embryonic Stem Cells and Obtaining of Spherical Aggregates

Mouse embryonic stem cells cultured in DMEM (with essential amino acids and LIF protein to maintain pluripotency) by incubation for 30 min with the iron oxide nanoparticles for concentrations ranging from [Fe]=0.1 mM to [Fe]=2 mM. The cell uptake per embryonic cell then ranges from 1 pg to 4 pg of iron per cell (see curve below). 50,000 to 250,000 cells were deposited on a glass substrate, 100 μm from the magnetic attractor. The magnetic force applied to the cells ranges between 50 and 200 pN. For all the conditions, spherically shaped cohesive aggregates are obtained.

1-18. (canceled)

19. A process for the aggregation of stem cells or of cells undergoing differentiation, carried out in the absence of three-dimensional support matrix, comprising:
a) bringing stem cells or cells undergoing differentiation into contact, in vitro or ex vivo, with magnetic particles, said bringing into contact making it possible to obtain magnetised cells, and
b) exposing said magnetised cells to a magnetic field, said exposure enabling the formation of a cell aggregate and being performed in the absence of a three-dimensional support matrix.

20. The process according to claim 19, in which the stem cells are mesenchymal stem cells (MSCs), embryonic stem cells (ESCs) or induced progenitor cells.

21. The process according to claim 19, in which the magnetic field is a focused field.

22. The process according to claim 19, in which step b) comprises exposing the magnetised cells to a magnetic field that forms one or more magnetic shapes.

23. The process according to claim 22, in which the magnetised cells are exposed to a magnetic shape and said shape is chosen from a spot, the tip or the end of a truncated needle, a line, a cross, a star, a ring, a disc, a circle, or several of one and/or the other of said shapes.

24. The process according to claim 19, said process being carried out without a centrifugation step and optionally in the absence of a differentiation factor.

25. A cell aggregate obtained by a process according to claim 19, wherein said aggregate comprises magnetised stem cells, magnetised stem cells undergoing differentiation, and/or magnetised cells in the process of differentiating, and, optionally, differentiated magnetised cells.

26. The cell aggregate according to claim 25, characterized in that it is obtained using a process employing 0.5, 1 or 2 million cells.

27. A process for preparing a tissue structure, comprising the implementation of a process for aggregation of cells in the absence of three-dimensional support matrix according to claim 19, optionally carried out in the absence of growth factor during the first day or the first 2, 3, 4, 5, 6, 7 or 8 days of the culturing step following the implementation of the aggregation process.

28. The process according to claim 27, said process further comprising culturing one or more cell aggregates obtained at the end of said aggregation process.

29. The process according to claim 27, wherein said process enables the preparation of a cartilaginous structure and is implemented in the absence of growth factor.

30. The process according to claim 29, wherein said process is implemented in the absence of TGFβ1, TGFβ3 and/or BMP-2, at least during the aggregation step.

31. The process according to claim 28, wherein the culturing step is carried out in the presence of a magnetic field created using one or more magnetic sources.

32. A tissue structure obtained using a process implemented in the absence of three-dimensional support matrix according to claim 29, comprising (i) differentiated magnetised cells and (ii) an extracellular matrix.

33. The tissue structure according to claim 32, wherein said tissue structure comprises (i) magnetic cartilaginous cells and (ii) a cartilaginous extracellular matrix.

34. The tissue structure according to claim 32, said structure being in the form of a tissue stick, patch, sheet or rod.

35. A method for visualising a tissue structure according to claim 32 comprising imaging said tissue structure.
